(19) United States
(12) Patent Application Publication
(10) Pub. No.: US 2009/0004114 A1
(43) Pub. Date: Jan. 1, 2009

(54) System and Method for Monitoring in Vivo Drug Release Using Overhauser-Enhanced Nmr

(75) Inventor: Johan Overweg, Uelzen (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/096,191

(22) PCT Filed: Nov. 15, 2006
(86) PCT No.: PCT/IB06/54273

§ 371 Date: Jun. 5, 2008

Related U.S. Application Data
(60) Provisional application No. 60/748,677, filed on Dec. 8, 2005.

(51) Int. Cl. A61K 49/18; A61K 49/06

(52) U.S. Cl. 424/9.3; 977/930

(57) ABSTRACT

Systems and methods for monitoring in vivo release of therapeutic and/or diagnostic agents, e.g., drugs, are provided. The disclosed systems and methods use a contrast agent and Overhauser-enhanced nuclear magnetic resonance (NMR) to monitor and/or measure the concentration and distribution of the contrast agent. Provided the contrast agent and the therapeutic/diagnostic agent have similar pharmaco-kinetics, the disclosed system/method may also be used to monitor and/or measure the concentration of such therapeutic/diagnostic agent (e.g., a drug), e.g., in the form of a volume-averaged signal and/or dynamic two-dimensional or three-dimensional images. In exemplary embodiments of the present disclosure, the therapeutic/diagnostic agent and the contrast agent are introduced to the body in an encapsulated form, e.g., within hollow nanoparticles.

The present disclosure is directed to a system and method for monitoring in vivo release of therapeutic and/or diagnostic agents, e.g., drugs, and more particularly, to the use of a contrast agent and Overhauser-enhanced nuclear magnetic resonance (NMR) to monitor and/or measure the concentration and distribution of the contrast agent. Provided the contrast agent and the therapeutic/diagnostic agent have similar pharmaco-kinetics, the disclosed system/method may also be used to monitor and/or measure the concentration of such therapeutic/diagnostic agent (e.g., a drug), e.g., in the form of a volume-averaged signal and/or dynamic two-dimensional or three-dimensional images. In exemplary embodiments of the present disclosure, the therapeutic/diagnostic agent and the contrast agent are introduced to the body in an encapsulated form, e.g., within hollow nanoparticles.

The drug delivery industry is involved in developing technologies that enhance and enable the use of chemical or biological compounds as therapeutic agents. Drug delivery systems are generally aimed at enhancing therapeutic effectiveness by controlling the rate, time, and location of release of a drug or drugs in the body. Among the issues of significance in evaluating drug delivery systems are safety, efficacy, ease of patient use, and patient compliance. Similarly, delivery systems for diagnostic agents and other clinically-relevant molecules and compounds.

Improved drug delivery offers pharmaceutical and biotechnology companies, competing in the pharmaceutical industry, a means of gaining a competitive advantage. Novel drug delivery technologies can accomplish this by improving the life cycle of existing drugs through advancements in safety, efficacy, and ease of use. Improved drug delivery can also enhance the eventual marketability of new compounds in the production pipeline. Advances in biotechnology have facilitated the development of a new generation of biopharmaceutical products based on proteins, peptides, and nucleic acids. However, these compounds present drug delivery challenges because they are often large, complex molecules, or small molecules that degrade rapidly in the bloodstream. Thus, if improved functionality of proteins and peptide-based products is to be realized, the development of innovative and novel drug delivery technologies becomes a prerequisite.

Medication can be delivered to a patient through a variety of methods, including oral ingestion, inhalation, transdermal diffusion, subcutaneous and intramuscular injection, parenteral administration, and implants. Oral drug delivery remains a preferred method of administering medication. Many currently marketed drug delivery products possess drawbacks. For example, conventional oral capsules and tablets have limited effectiveness in providing controlled drug delivery, often resulting in drug release that is too rapid and thus causing incomplete absorption of the drug, irritation of the gastrointestinal tract, and other side effects. Additionally, capsules and tablets generally cannot provide localized therapy.

The effectiveness of inhalation drug delivery products is often limited by the poor efficiency of pulmonary devices and the difficulty of administering high doses of certain drugs. Transdermal patches are often inconvenient to apply, can be irritating to the skin, and the rate of release can be difficult to control. Many drugs, especially large-molecule compounds, require parenteral injection delivery, which is often painful for patients and usually requires clinician administration (which can increase cost). Implants generally are administered in a hospital or physician s office and frequently are not suitable for home use. Thus, the increasing need to deliver medication and other agents to patients more efficiently and with fewer side effects has accelerated the development of new drug delivery systems.

Magnetic resonance imaging (MRI) is a diagnostic technique is a non-invasive technique that does not involve exposing the patient under study to potentially harmful radiation. Electron spin resonance enhanced MRI, which may be termed Overhauser MRI (OMRI), is a method of MRI in which enhancement of the magnetic resonance signals from which images may be generated is achieved by virtue of dynamic nuclear polarization, i.e., the Overhauser effect. The Overhauser effect occurs on VHF stimulation of an electron spin resonance (ESR) transition in a magnetic, usually paramagnetic, material. OMRI techniques have been described in the literature, e.g., EP-A-296833, EP-A-361551, WO-A-90/13047, J. Mag. Reson. 76:366-370 (1988), EP-A-302742, Society for Magnetic Resonance in Medicine (SMRM) 9:619 (1990), SMRM 6:24 (1987), SMRM 7:1094 (1988), SMRM 8:329 (1989), U.S. Pat. No. 4,719,425, SMRM 8:816 (1989), Mag. Reson. Med. 14:140-147 (1990), SMRM 9:617 (1990), SMRM 9:612 (1990), SMRM 9:121 (1990), GB-A-2227095, DE-A-4042212 and GB-A-2220269.

U.S. Pat. No. 5,479,925 (Dumoulin) discloses an imaging system for obtaining vessel-selective NMR angiographic images of a subject; U.S. Pat. No. 5,263,482 (Leunbach) discloses a method of and apparatus for thermographic imaging involving the use in OMRI of a paramagnetic contrast agent having a temperature dependent transition in its ESR spectrum; and U.S. Pat. No. 6,311,086 (Ardenkjaer-Larsen et al.) discloses a method of MR investigation of a sample that involves placing an OMRI contrast agent and an MR imaging agent in a uniform magnetic field, exposing the composition to a first radiation of a frequency selected to excite electron spin transitions in the OMRI contrast agent, separating the OMRI contrast agent from the MR imaging agent, administering the MR imaging agent to a sample, exposing the sample to a second radiation of a frequency selected to excite nuclear spin transitions, detecting magnetic resonance signals from the sample, and generating an image or dynamic flow data from the detected signals.

In basic in vivo OMRI techniques, the imaging sequence generally involves initially irradiating a subject placed in a uniform magnetic field (the primary magnetic field, B₀) with radiation, usually VHF radiation, of a frequency selected to excite a narrow linewidth ESR transition in an OMRI contrast agent which is in, or has been administered to, the subject. Dynamic nuclear polarization results in an increase in the population difference between the excited and ground nuclear spin states of selected nuclei, i.e. those nuclei, generally protons, which are responsible for the magnetic resonance signals. Since MR signal intensity is proportional to this population difference, the subsequent stages of each imaging sequence, performed essentially as in conventional MRI techniques, result in larger amplitude MR signals being detected. OMRI contrast agents which exhibit an ESR transition able to couple with an NMR transition of the MR imaging nuclei may be naturally present within the subject or may be administered thereto.

To be successful as an in vivo OMRI contrast agent in conventional methods of OMRI, a material must exhibit physiological tolerability. This factor alone imposes a severe limitation on the OMRI contrast agents which prove to be of diagnostic utility. Organic free radicals, for example, are frequently unstable in physiological conditions or have very short half-lives leading to toxicity problems. Indeed, radicals found to give excellent Overhauser enhancement factors in vitro frequently cannot be used diagnostically due to physiological incompatibility.

Despite efforts to date, a need remains for effective systems and methods for in vivo measurement of therapeutic and/or diagnostic agents. More particularly, a need remains for non-invasive systems and methods for monitoring and/or measuring in vivo delivery of therapeutic and/or diagnostic agents. Still further, a need remains for monitoring and/or measuring the concentration and distribution of therapeutic and/or diagnostic agents in vivo. These and other needs are satisfied by the systems and methods disclosed herein.

Systems and methods for monitoring and/or measuring in vivo release of therapeutic and/or diagnostic agents are provided herein. The disclosed systems and methods are particularly advantageous for monitoring and/or measuring the in vivo release of drugs and other therapeutic agents. According to exemplary embodiments of the present disclosure, the therapeutic and/or diagnostic agent is introduced with a contrast agent for an in vivo application, e.g., delayed release/time release of the therapeutic/diagnostic agent. An Overhauser-enhanced NMR is advantageously employed to monitor and/or measure the concentration and distribution of the contrast agent. According to further preferred implementations of the present disclosure, a contrast agent is selected that has similar pharmaco-kinetics relative to the therapeutic/diagnostic agent. By selecting a contrast agent having comparable pharmaco-kinetic properties, the disclosed system and method are advantageously able to monitor and/or measure the concentration/distribution of such therapeutic/diagnostic agent, e.g., in the form of a volume-averaged signal and/or dynamic two-dimensional or three-dimensional images. As noted herein, the therapeutic/diagnostic agent and the contrast agent may be advantageously introduced to the body in an encapsulated form, e.g., within hollow nanoparticles.

According to exemplary embodiments of the present disclosure, therapeutic and/or diagnostic agents are encapsulated within a delivery medium, e.g., hollow nanoparticles, together with an appropriate contrast agent. The encapsulated delivery medium is then introduced into the body, e.g., by injection, oral administration or the like. The delivery medium advantageously becomes concentrated in the organ or region of the body of interest, e.g., the body organ to which an encapsulated drug is to be delivered and/or for which the encapsulated drug is active. Techniques for achieving localized concentration of delivery media in regions/organs of the body are well known to persons skilled in the art, and the disclosed systems/methods may be used in conjunction with any such delivery regimen.

While the delivery media, e.g., hollow nanoparticles, remain substantially intact, the concentration and distribution of the hollow nanoparticles in a volume of tissue are mapped by ESR imaging. The ESR mapping is generally undertaken by irradiating the body/patient at the frequency of the electron transition of the encapsulated contrast agent, e.g., a triarylmethyl (trityl radical) structure. The emitted signal after excitation is measured. Of note, the measured signal generally increases in an approximately linear fashion relative to increases in the amount of the trityl radical, independent of whether the contrast agent is encapsulated or released from the delivery medium. No significant Overhauser effect or enhancement is noted in this initial ESR reading because the trityl radicals are concentrated in a relatively small volume fraction, e.g., the interior volume of the hollow nanoparticles that are functioning as a delivery medium for the therapeutic and/or diagnostic agent.

After the initial ESR measurement, the therapeutic and/or diagnostic agent is typically delivered from the delivery medium by breakdown and/or disintegration of the encapsulating medium, whether in whole or in part. Thus, in an exemplary embodiment of the present disclosure, the encapsulating medium takes the form of hollow nanoparticles and the encapsulated therapeutic and/or diagnostic agent (as well as the encapsulated contrast agent) is released by rupturing the nanoparticles walls. Various forces may be used to release the encapsulated agents from the delivery medium, e.g., focused ultrasound energy and/or RF heating. Alternatively, internal anatomical forces may be relied upon to release the encapsulated agents, as is well known in the art.

Once the encapsulated agents, i.e., the therapeutic/diagnostic agents and the contrast agent, are released from the delivery medium, further measurements are made using NMR/MRI techniques. Thus, by saturating the ESR transition for a period of time, the longitudinal polarization of the protons associated with the contrast agent is modified. With a concentration of the trityl in the 1-10 millimolar range, the proton polarization can be increased by a factor of 10-100. For purposes of NMR imaging, the NMR signal changes in a manner that is roughly proportional to such proton polarization. Of note, the NMR signal does not increase linearly with trityl concentration; rather, the enhancement reaches a saturation level with increasing trityl radical concentration. This non-linear response is particularly advantageous for purposes of the systems and methods of the present disclosure.

More particularly, for purposes of the in vivo measurements associated with the systems and methods of the present disclosure, the encapsulated therapeutic/diagnostic agents and the contrast agent are dispersed into body tissue after release from the delivery medium. As the water associated with the tissue comes into contact with the contrast agent, e.g., the trityl radicals associated therewith, a large NMR signal enhancement is generally observed. As time passes and the contents of the delivery medium are further deployed into the surrounding body tissue, the agents are generally washed out and/or metabolized, thereby reducing the Overhauser signal. Thus, the NMR signal responds and reflects the in vivo activities associated with the contrast agent and, to the extent the pharmaco-kinetics of the therapeutic/diagnostic agents are similar to the contrast agent, the NMR signal can also be used to monitor/measure the concentration and/or distribution of the released therapeutic/diagnostic agent, e.g., a drug.

The systems and methods of the present disclosure may be employed to measure the in vivo behavior of a deployed therapeutic/diagnostic agent in a variety of ways. For example, the NMR results described herein may be used to generate a volume-averaged signal which is generally useful, for example, to investigate/monitor the dynamics of drug release. Alternatively, the NMR results may be used to generate two-dimensional or three-dimensional images that show the distribution of the contrast agent and, assuming comparable pharmaco-kinetic properties, the associated therapeutic and/or diagnostic agent. The 2D/3D images are advantageously generated in a dynamic manner. Beyond the NMR results collected post-release of the encapsulated agents, the ESR signal may be used to measure/monitor the total amounts of contrast agent (e.g., based on the trityl radical) and/or therapeutic/diagnostic agent in the anatomical region of interest.

According to exemplary embodiments of the disclosed systems and methods, RF energy is used to release the encapsulated agents from the delivery medium, e.g., hollow nanoparticles. The RF power required to release the agents from the delivery medium may be advantageously selected so as to approximately equal the ESR excitation associated with Overhauser NMR. Additional features, functions and benefits associated with the disclosed systems and methods will be apparent from the description which follows.

To assist those of skill in the relevant art in using the disclosed systems and methods, reference is made to the accompanying figure, wherein:

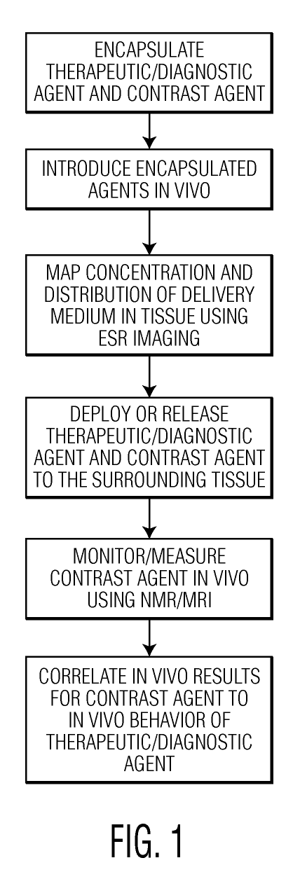

FIG. 1 is a schematic flowchart setting forth exemplary process steps for monitoring and/or measuring in vivo delivery of therapeutic and/or diagnostic agents; and

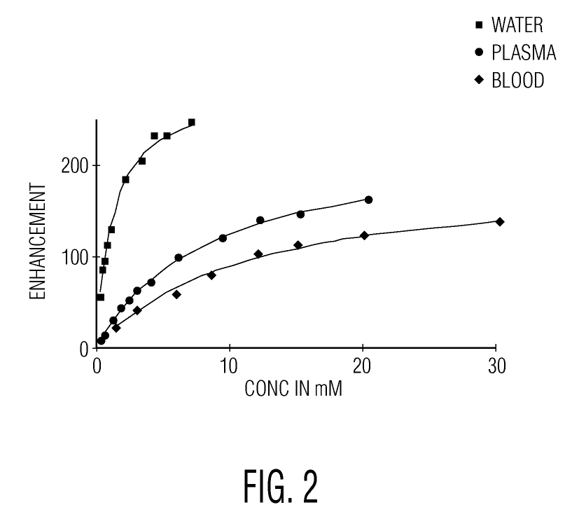

FIG. 2 is a plot of DNP enhancement versus trityl concentration for three media (water, plasma and blood) at 37° C.

The present disclosure provides systems and methods for monitoring and/or measuring in vivo release of therapeutic and/or diagnostic agents, e.g., drugs and other therapeutic agents. The therapeutic and/or diagnostic agent is typically introduced with a contrast agent and an Overhauser-enhanced NMR is employed to monitor and/or measure the concentration and distribution of the contrast agent. The contrast agent may be selected so as to exhibit similar pharmaco-kinetics relative to the therapeutic/diagnostic agent encapsulated therewith, thereby facilitating the concentration/distribution of the therapeutic/diagnostic agent to be simultaneously achieved. Various imaging techniques may be employed to monitor/measure in vivo concentrations and/or distributions of the agents, e.g., a volume-averaged signal and/or dynamic two-dimensional or three-dimensional images.

With reference to the flowchart of FIG. 1, a therapeutic/diagnostic agent and a contrast agent are initially encapsulated within a delivery medium. In exemplary embodiments of the present disclosure, the foregoing agents are encapsulated within hollow nanoparticles that are appropriate for clinical applications. Alternative encapsulation materials and encapsulation techniques may be employed without departing from the spirit or scope of the present disclosure, e.g., conventional microencapsulation techniques.

Contrast agents useful in the disclosed systems and methods are well known in the literature. For example, suitable contrast agents are disclosed in the following patent publications: WO-A-88/10419; WO-A-90/00904; WO-A-91/12024; WO-A-96/39367; WO-A-93/02711 and UK Patent Application No. 9605482.0. Nanoparticle technology for encapsulation of materials/agents of the type disclosed herein is also well known to persons skilled in the art. For example, U.S. Pat. Nos. 6,632,671 and 6,602,932 disclose exemplary techniques for nanoparticles encapsulation of materials.

The encapsulated delivery medium is then introduced into the body, e.g., by injection, oral administration or the like. The manner of administration of the delivery medium is not critical to the present disclosure. Generally, the delivery medium concentrates in the organ or region of interest, e.g., within a body organ or body region to which an encapsulated drug is to be delivered and/or for which the encapsulated drug is active. Techniques for achieving localized concentration of delivery media in regions/organs of the body are well known to persons skilled in the art, and the disclosed systems/methods may be used in conjunction with any such delivery regimen.

After introducing the delivery medium to the body, the concentration and distribution of the hollow nanoparticles in a volume of tissue are mapped by ESR imaging. ESR mapping is generally undertaken by irradiating the body/patient at the frequency of the electron transition of the encapsulated contrast agent, e.g., a triarylmethyl (trityl radical) structure, while the delivery medium remains substantially intact, and measuring the emitted signal after excitation. The signal response is generally linear with respect to increases in the presence of a trityl radical (contrast agent), independent of whether the contrast agent is encapsulated or released from the delivery medium. No significant Overhauser effect or enhancement is observed in this initial ESR reading because the trityl radicals are concentrated in a relatively small volume fraction, e.g., the interior volume of the hollow nanoparticles. ESR imaging is generally undertaken using conventional ESR instrumentation, e.g., ESR systems that include a whole-body magnet operated in a field-cycle mode to avoid excess power deposition. The selection and operation of ESR equipment for purposes of the disclosed systems and methods is well within the skill of persons possessing ordinary skill in the relevant field.

After the initial ESR measurement, the therapeutic and/or diagnostic agent(s) are typically delivered from the delivery medium by breakdown and/or disintegration of the encapsulating delivery medium. The delivery medium may be disintegrated in whole or in part, thereby releasing the agents contained therein to the surrounding tissue. In an exemplary embodiment of the present disclosure, the encapsulating delivery medium includes a plurality of hollow nanoparticles within which are encapsulated therapeutic and/or diagnostic agents. Also encapsulated within the delivery medium is a contrast agent. The encapsulated agents are released from the hollow nanoparticles by rupturing the nanoparticles walls. Various forces may be used to release the encapsulated agents from the delivery medium, e.g., focused ultrasound energy and/or RF heating. Alternatively, internal anatomical forces may be relied upon to release the encapsulated agents, as is well known in the art. According to exemplary embodiments of the disclosed systems and methods, RF energy is used to release the encapsulated agents from the delivery medium, e.g., hollow nanoparticles. The RF power required to release the agents from the delivery medium may be advantageously selected so as to approximately equal the ESR excitation associated with Overhauser NMR.

Once the encapsulated agents, i.e., the therapeutic/diagnostic agents and the contrast agent, are released from the delivery medium, further measurements are made using NMR/MRI techniques. The encapsulated therapeutic/diagnostic agents and the contrast agent are dispersed into body tissue after release from the delivery medium, thereby bringing the water associated with tissue into contact with the contrast agent, e.g., the trityl radicals associated therewith. Based on the interaction between the contrast agent and the water of the tissue, a large NMR signal enhancement is generally observed. Over time, the agents are generally washed out and/or metabolized, thereby reducing the Overhauser signal. Thus, the NMR signal reflects the in vivo fall-off in contrast agent concentration and, to the extent the pharmaco-kinetics of the therapeutic/diagnostic agents are similar to the contrast agent, the NMR signal can also be used to monitor/measure the concentration and/or distribution of the released therapeutic/diagnostic agent, e.g., a drug.

Of note, it is contemplated according to the present disclosure that the functionalities associated with the therapeutic agent/molecule and the contrast agent could be incorporated into a single molecule, ligand, substrate, composition or the like. By combining such functionalities into a single molecule, clinical and/or functional advantages may be derived. For example, issues associated with potential differences in pharmaco-kinetic properties between the therapeutic agent and the contrast agent would be eliminated by combining such functionalities into a single molecule. Similarly, any potential issues associated with dosing, clinical delivery and the like could be obviated by combining the therapeutic and contrast functionalities into a single molecule, ligand, substrate, composition or the like.

In making the NMR measurements described herein, the ESR transition is typically saturated for a period of time and the longitudinal polarization of the protons associated with the contrast agent is modified. With a concentration of the trityl radical in the 1-10 millimolar range, proton polarization is typically increased by a factor of 10-100. For purposes of NMR imaging, the NMR signal changes in a manner that is roughly proportional to such proton polarization. The NMR signal does not increase linearly with trityl concentration; rather, the enhancement reaches a saturation level with increasing trityl radical concentration, i.e., providing a non-linear response. The plots of FIG. 2 illustrate the non-linear relationship between DNP enhancement (dynamic nuclear polarization enhancement) and trityl concentration in three media: water, plasma and blood at 37° C.

The concentration/distribution measurements generated by the disclosed systems and methods may take a variety of forms. For example, the NMR results described herein may be used to generate a volume-averaged signal which is generally useful, for example, to investigate/monitor the dynamics of drug release. Alternatively, the NMR results may be used to generate two-dimensional or three-dimensional images that show the distribution of the contrast agent and, assuming comparable pharmaco-kinetic properties, the associated therapeutic and/or diagnostic agent. The 2D/3D images are advantageously generated in a dynamic manner. Beyond the NMR results collected post-release of the encapsulated agents, the ESR signal may be used to measure/monitor the total amounts of contrast agent (e.g., based on the trityl radical) and/or therapeutic/diagnostic agent in the anatomical region of interest.

Although the disclosed systems and methods have been described with reference to exemplary embodiments thereof, the present disclosure is not limited to such exemplary implementations. For example, although the present disclosure is primarily described with reference to human applications, the systems and methods disclosed herein may be employed with equal benefit to other animal systems. Thus, the systems and methods of the present disclosure is susceptible to many variations, modifications and/or enhancements without departing from the spirit or scope hereof, and the present disclosure is expressly intended to encompass such variations, modifications and/or enhancements.

1. A system for monitoring the release of one or more agents in vivo, comprising:
a. a delivery medium that includes at least one diagnostic or therapeutic agent and at least one contrast agent; and
b. a source of ESR and of NMR irradiation;
wherein the delivery medium is adapted to release the at least one diagnostic or therapeutic agent and at least one contrast agent in vivo; and
wherein the ESR and NMR irradiation function to monitor the release of the at least one contrast agent in vivo.

2. A system according to claim 1, wherein the delivery medium includes hollow nanoparticles.

3. A system according to claim 1, wherein the at least one diagnostic or therapeutic agent is a drug.

4. A system according to claim 1, wherein the at least one diagnostic or therapeutic agent and the at least one contrast agent have similar pharmaco-kinetics.

5. A system according to claim 1, wherein the sources of ESR and NMR irradiation are further adapted to monitor the release of the at least one diagnostic or therapeutic agent.

6. A system according to claim 1, further comprising means for generating a signal or image corresponding, in whole or in part, to the release of the at least one contrast agent in vivo.

7. A system according to claim 6, wherein the signal or image further corresponds, in whole or in part, to the release of the at least one diagnostic or therapeutic agent in vivo.

8. A system according to claim 6, wherein the signal or image is selected from the group consisting of volume-averaged signal, a two-dimensional image, a three-dimensional image, and combinations thereof.

9. A system according to claim 1, wherein the source of ESR irradiation is further adapted to supply energy to the delivery medium to cause a release of the agents contained therein.

10. A system according to claim 9, wherein the source of ESR irradiation is adapted to deliver RF power that is effective to release the agents from the delivery medium and to cause ESR excitation for purposes of Overhauser NMR measurement.

11. A system according to claim 1, wherein the at least one diagnostic or therapeutic agent and the at least one contrast agent are included in a single molecule, ligand, substrate, composition or the like.

12. A method for monitoring the release of one or more agents in vivo, comprising:
a. introducing a delivery medium in vivo, the delivery medium including at least one diagnostic or therapeutic agent and at least one contrast agent;
b. causing release of the at least one diagnostic or therapeutic agent and the at least one contrast agent from the delivery medium in vivo; and
c. delivering irradiation from ESR and NMR sources that is effective to monitor release of the at least one contrast agent in vivo.

13. A method according to claim 12, wherein the delivery medium includes hollow nanoparticles.

14. A method according to claim 12, wherein the delivery medium is delivered in vivo by injection or oral administration.

15. A method according to claim 12, wherein release of the at least one diagnostic or therapeutic agent and the at least one contrast agent is caused by rupturing the wall of the delivery medium.

16. A method according to claim 15, wherein rupture of the delivery medium wall is caused by RF power supplied by the ESR source.

17. A method according to claim 12, wherein ESR imaging is effected prior to release of the at least one diagnostic or therapeutic agent and the at least one contrast agent from the delivery medium in vivo.

18. A method according to claim 12, wherein the pharmaco-kinetic properties of the at least one diagnostic or therapeutic agent and the at least one contrast agent are approximately the same, and wherein the irradiation from the ESR and NMR sources is further effective to monitor release of the at least one diagnostic or therapeutic agent in vivo.

19. A method according to claim 12, further comprising generating a signal or image with respect to release of the agent(s).

20. A method according to claim 19, wherein the signal or image is selected from the group consisting of volume-averaged signal, a two-dimensional image, a three-dimensional image, and combinations thereof.

21. A method according to claim 12, wherein the wherein the at least one diagnostic or therapeutic agent and the at least one contrast agent are included in a single molecule, ligand, substrate, composition or the like.
